SEQUENCE LISTING (33 sequences)

SEQ ID NO: 1 - protein, 943 aa, Artificial Sequence
  Description of Artificial Sequence Synthetic polypeptide
MASLVVLIDGHSLAYRAYYALLRQGLQTRKGEPTHAVYGFTSMLLRVWREHQPTYMAVAFDVGRTFRHEAFAEYKATRAERPSDFDAQLARIRQIIAAFAIPALTAEGFEADDVIGTAATQALARGMEVLIVTGDTDTFQLIRPGLRVLTSRRHFDDVVIYDVEAIRERYGLEPSQLVDLKALTGDPSDNIPGVRGIGERTATELLKRYGSLENLYAHLDEIQPERVRRLLEAGREQAFLSKRLAQIRTDVPLTIDWEACRVGRYDRAAVLALFQELEFRSLIPRLPDGAAPEEAPAAPEAASGPTGQPMLFPEPLGPAVTRPIAMPPLRAEGLPRPTEATVVTDEAGLAALQEALARAPRFAFDVETDTLAPMRANPVGLAFAVEDGRGYYVPVGHRDGSPQLPLPRVLDALRPFLADPGRPKVAHHAKYDMLVLARHGVMVQGLAFDTMVAEWLLNPTAHGLSLKHLAWLRLGVEMTTIEQLIGKGKGQRSFADVPTSAAAPYAAADADLTLRIARVQEEELRRQGLWDLFAQVEIPLIPVLVRMELAGVLIDVALLQQMSKEFERRIQALAQEIYRWVGYAFNLDSPRQLSDALFGKLQLPRANVPRTGTGMYSTSSEVLEGLRGAHPVIDLILEYRQLAKLKSTYIDALPRMIHPATGRIHPSYHQTGTVTGRISASDPNIQNIPIRTELGKQIRRAFIAPPGSVLLSADYSQIELRILAHITRDPGLIAAFQAGEDIHRATAARAFGIPPEQVTEEQRNFAKRINYGLIYGMSAHGLAQQLGISRREAERFIEQYFAAFPRVRAYIEAIKARAARQGYVETLLGRRRYFPELKPAGGEGLPRRVPEAVRRAAEREAINAPIQGSAADILKLAMIRIDRAMQEQGLRTRMIMQVHDELVFEVPEDEVEAVQGLVAEAMRTAYPLAVPLEVELHMGPCWS

SEQ ID NO: 2 - protein, 654 aa, Artificial Sequence
  Description of Artificial Sequence Synthetic polypeptide
MAPEEAPAAPEAASGPTGQPMLFPEPLGPAVTRPIAMPPLRAEGLPRPTEATVVTDEAGLAALQEALARAPRFAFAVETDTLAPMRANPVGLAFAVEDGRGYYVPVGHRDGSPQLPLPRVLDALRPFLADPGRPKVAHHAKYDMLVLARHGVMVQGLAFDTMVAEWLLNPTAHGLSLKHLAWLRLGVEMTTIEQLIGKGKGQRSFADVPTSAAAPYAAADADLTLRIARVQEEELRRQGLWDLFAQVEIPLIPVLVRMELAGVLIDVALLQQMSKEFERRIQALAQEIYRWVGYAFNLDSPRQLSDALFGKLQLPRANVPRTGTGMYSTSSEVLEGLRGAHPVIDLILEYRQLAKLKSTYIDALPRMIHPATGRIHPSYHQTGTVTGRISASDPNIQNIPIRTELGKQIRRAFIAPPGSVLLSADYSQIELRILAHITRDPGLIAAFQAGEDIHRATAARAFGIPPEQVTEEQRNFAKRINYGLIYGMSAHGLAQQLGISRREAERFIEQYFAAFPRVRAYIEAIKARAARQGYVETLLGRRRYFPELKPAGGEGLPRRVPEAVRRAAEREAINAPIQGSAADILKLAMIRIDRAMQEQGLRTRMIMQVHDELVFEVPEDEVEAVQGLVAEAMRTAYPLAVPLEVELHMGPCWS

SEQ ID NO: 3 - protein, 6 aa, Artificial Sequence
  Description of Artificial Sequence Synthetic peptide
HHHHHH

SEQ ID NO: 4 - DNA, 30 bp, Artificial Sequence
  Description of Artificial Sequence Synthetic primer
gagccatattcaacgggaaacgtcttgctc

SEQ ID NO: 5 - DNA, 21 bp, Artificial Sequence
  Description of Artificial Sequence Synthetic primer
gagccatattcaacgggaaac

SEQ ID NO: 6 - DNA, 15 bp, Artificial Sequence
  Description of Artificial Sequence Synthetic primer
gagccatattcaacg

SEQ ID NO: 7 - DNA, 26 bp, Artificial Sequence
  Description of Artificial Sequence Synthetic primer
gatcctggtatcggtctgcgattccg

SEQ ID NO: 8 - DNA, 19 bp, Artificial Sequence
  Description of Artificial Sequence Synthetic primer
gatcctggtatcggtctgc

SEQ ID NO: 9 - DNA, 15 bp, Artificial Sequence
  Description of Artificial Sequence Synthetic primer
agatcctggtatcgg

SEQ ID NO: 10 - DNA, 18 bp, Artificial Sequence
  Description of Artificial Sequence Synthetic primer
tggaccccaaaatcagcg

SEQ ID NO: 11 - DNA, 18 bp, Artificial Sequence
  Description of Artificial Sequence Synthetic primer
ggaacgccttgtcctcga

SEQ ID NO: 12 - DNA, 41 bp, Artificial Sequence
  Description of Artificial Sequence Synthetic primer
ccactgcgttctccattctggtaaatgcaccccgcattacg

SEQ ID NO: 13 - DNA, 40 bp, Artificial Sequence
  Description of Artificial Sequence Synthetic primer
caacgtcggccccaaggtttggtcttccttgccatgttga

SEQ ID NO: 14 - DNA, 21 bp, Artificial Sequence
  Description of Artificial Sequence Synthetic primer
tgaatctgagggtccaccaaa

SEQ ID NO: 15 - DNA, 22 bp, Artificial Sequence
  Description of Artificial Sequence Synthetic primer
acccaataatactgcgtcttgg

SEQ ID NO: 16 - DNA, 15 bp, Artificial Sequence
  Description of Artificial Sequence Synthetic primer
tattgtctgtgccac

SEQ ID NO: 17 - DNA, 15 bp, Artificial Sequence
  Description of Artificial Sequence Synthetic primer
aaactatcggccttg

SEQ ID NO: 18 - DNA, 32 bp, Artificial Sequence
  Description of Artificial Sequence Synthetic primer
cacacgaccagtaataatttcaggtcagaagg

SEQ ID NO: 19 - DNA, 30 bp, Artificial Sequence
  Description of Artificial Sequence Synthetic primer
gacgattgagcgtcaacaatattaccgcca

SEQ ID NO: 20 - DNA, 17 bp, Artificial Sequence
  Description of Artificial Sequence Synthetic primer
gccaacagagatagaac

SEQ ID NO: 21 - DNA, 18 bp, Artificial Sequence
  Description of Artificial Sequence Synthetic primer
gtaggtatttccatgagc

SEQ ID NO: 22 - DNA, 18 bp, Artificial Sequence
  Description of Artificial Sequence Synthetic primer
gcagcacatccccctttc

SEQ ID NO: 23 - DNA, 19 bp, Artificial Sequence
  Description of Artificial Sequence Synthetic primer
cgtgcatctgccagtttga

SEQ ID NO: 24 - DNA, 38 bp, Artificial Sequence
  Description of Artificial Sequence Synthetic primer
ggctgcgcaactgttgggaaccagctggcgtaatagcg

SEQ ID NO: 25 - DNA, 39 bp, Artificial Sequence
  Description of Artificial Sequence Synthetic primer
ctttgcctggtttccggcacacgacagtatcggcctcag

SEQ ID NO: 26 - DNA, 17 bp, Artificial Sequence
  Description of Artificial Sequence Synthetic primer
atcggtgcgggcctctt

SEQ ID NO: 27 - DNA, 19 bp, Artificial Sequence
  Description of Artificial Sequence Synthetic primer
cagaagcggtgccggaaag

SEQ ID NO: 28 - DNA, 17 bp, Artificial Sequence
  Description of Artificial Sequence Synthetic primer
gcgtgatggtgggcatg

SEQ ID NO: 29 - DNA, 19 bp, Artificial Sequence
  Description of Artificial Sequence Synthetic primer
tctgggtcatcttctcgcg

SEQ ID NO: 30 - DNA, 39 bp, Artificial Sequence
  Description of Artificial Sequence Synthetic primer
tgccgtgctcgatggggtagaaggattcctatgtgggcg

SEQ ID NO: 31 - DNA, 36 bp, Artificial Sequence
  Description of Artificial Sequence Synthetic primer
ctcccgaggagcaccccggttggccttggggttcag

SEQ ID NO: 32 - DNA, 18 bp, Artificial Sequence
  Description of Artificial Sequence Synthetic primer
gcctctcttgctctgggc

SEQ ID NO: 33 - DNA, 17 bp, Artificial Sequence
  Description of Artificial Sequence Synthetic primer
ctgctgaccgaggcccc

(19) United States
(12) Patent Application Publication
(10) Pub. No.: US 2024/0150735 A1
(43) Pub. Date: May 9, 2024

(54) POLYMERASES FOR ISOTHERMAL NUCLEIC ACID AMPLIFICATION

(71) Applicant: Amazon Technologies, Inc., Reno, NV (US)

(72) Inventors: Guillermo Daniel REPIZO, San Francisco, CA (US); Maria Julia LARA, Rosario (AR); Carla Alejandra GIMENEZ, Buenos Aires (AR); Antonela Rocio PALACIOS, Rosario (AR); Alejandro PEZZA, Rosario (AR); Ivana Lorna PARCERISA, Rosario (AR); Matias Ezequiel CABRUJA, Rosario (AR)

(21) Appl. No.: 17/790,169

(22) PCT Filed: Jun. 21, 2022
(86) PCT No.: PCT/US2022/034319

§ 371 Date: Dec. 29, 2022

Related U.S. Application Data
(60) Provisional application No. 63/212,896, filed on Jun. 21, 2021.

(51) Int. Cl. C12N 9/12; C12N 15/63; C12Q 1/6844

(52) U.S. Cl. CPC C12N 9/1252; C12N 15/63; C12Q 1/6844; C12Y 207/07007

(57) ABSTRACT

Provided herein are recombinant polymerases that are suitable for nucleic acid amplification assays. The recombinant polymerases disclosed herein are useful in many recombinant DNA techniques, in particular nucleic acid amplification. Also provided herein are methods of producing the recombinant polymerases.

### 1. CROSS-REFERENCE TO RELATED APPLICATIONS

This application claims the benefit of U.S. Provisional Patent Application 63/212,896, filed Jun. 21, 2021, the entire contents of which are incorporated herein by reference.

### 2. SEQUENCE LISTING

This application contains an ASCII copy of a Sequence Listing in computer readable form. The computer readable form is incorporated herein by reference. Said ASCII copy, created on Jun. 20, 2022, is named 146401_091829_SL.txt and is 21,150 bytes in size.

### 3. BACKGROUND

Methods to amplify DNA and/or RNA are important for diagnostic purposes. These methods commonly use enzymes to synthesize biomolecules in vitro. One particularly useful class of enzymes is the polymerases, which can catalyze the polymerization of biomolecules (e.g., nucleotides or amino acids) into biopolymers (e.g., nucleic acids or peptides). For example, polymerases that can polymerize nucleotides into nucleic acids, particularly in a template-dependent fashion, are useful in recombinant DNA technology and nucleic acid sequencing applications.

Isothermal amplification has emerged as a useful method for the amplification of nucleic acids. Isothermal amplification rapidly and efficiently accumulates nucleic acid sequences at a constant temperature. However, the number of polymerase enzymes that are suitable for isothermal amplification technologies is limited. In particular, many of the known DNA polymerases have activity at narrow temperatures. For example, the E. coli Klenow fragment DNA polymerase exhibits activity at a low and narrow temperature range.

Thus, there is a need for new polymerase enzymes that are suitable for nucleic acid amplification assays.

### 4. SUMMARY

The inventors have developed novel recombinant polymerases that are particularly advantageous for nucleic acid amplification assays, such as isothermal amplification. For example, a particular DNA polymerase is active at a range of temperatures (e.g., about 51° C. to about 85° C., such as, for example, about 52° C. up to about 65° C.).

Accordingly, this disclosure relates to recombinant polymerases. The recombinant polymerase can be a DNA polymerase. The recombinant polymerase can comprise an amino acid sequence having at least about 70% sequence identity to SEQ ID NO: 1 or SEQ ID NO: 2. SEQ ID NO: 1 corresponds to the full-length recombinant DNA polymerase. SEQ ID NO: 2 corresponds to the C-terminus fragment of the recombinant polymerase. The recombinant polymerase can comprise an amino acid sequence having at least about 90% sequence identity to SEQ ID NO: 1 or SEQ ID NO: 2. The recombinant polymerase can comprise an amino acid sequence having at least 99% sequence identity to SEQ ID NO: 2.

In embodiments, the recombinant polymerase can be active with a single-stranded DNA (ssDNA) template. In embodiments, the recombinant polymerase can be active with a double-stranded DNA (dsDNA) template. In embodiments, the recombinant polymerase can be active with an RNA template, i.e., the recombinant polymerase can have reverse transcriptase or retrotranscriptase activity.

Generally, the recombinant polymerase disclosed herein is capable of 3′-5′ exonuclease activity. The recombinant polymerase may lack 5′-3′ exonuclease activity. The recombinant polymerase can have activity at a temperature of about 52° C. to about 65° C. The recombinant polymerase is particularly suitable for use in isothermal amplification.

The recombinant polymerase disclosed herein can be capable of 3′-5′ exonuclease activity and/or 5′-3′ exonuclease activity. The recombinant polymerase may lack 5′-3′ exonuclease activity and/or 3′-5′ exonuclease activity. The recombinant polymerase can have activity at a temperature of about 51° C. to about 85° C., such as about 52° C. to about 65° C., or about 70° C. to about 80° C.

Also described herein are compositions comprising the recombinant polymerase.

Provided herein is an isolated nucleic acid that comprises a nucleotide sequence that encodes the recombinant polymerase, an expression vector comprising the isolated nucleic acid, and a host cell comprising the expression vector.

In addition, the disclosure provides kits comprising the recombinant polymerase disclosed herein, a buffer, and optionally a divalent metal, an extension nucleotide, a primer, a probe, a detergent, a detection agent, a dye, a fluorescent molecule, an anticoagulant, nucleoside triphosphates, or a cell lysis agent.

For example, the kits can optionally comprise a divalent metal, an extension nucleotide, a primer, a probe, a detergent, a detection agent, a dye, a fluorescent molecule, an anticoagulant, nucleoside triphosphates, a cell lysis agent, a salt (e.g., NaCl, KCl, sodium acetate (NaAc), or KAc), MgSO₄, dimethyl sulfoxide (DMSO), Triton X100, Tween 20, betaine, urea, bovine serum albumin (BSA), taurine, dithiothreitol (DTT), tris(2-carboxyethyl)phosphine (TCEP), trehalose, NH₄SO₄, tetramethyl ammonium chloride (TMAC), tetra-n-propylammonium chloride (TPAC), polyethylene glycol (PEG), and/or 1,2-propanediol.

The disclosure also relates to methods of amplifying a DNA template. The method can comprise contacting the DNA template with the recombinant polymerase, one or more primers that specifically bind to the DNA template, and extension nucleotides to form a reaction mixture; and incubating the reaction mixture under conditions permitting extension of the one or more primers by the recombinant polymerase with the DNA template for the incorporation of the extension nucleotides.

The disclosure also relates to methods of amplifying a nucleic acid template. The method can comprise contacting the template with the recombinant polymerase, one or more primers that specifically bind to the template, and extension nucleotides to form a reaction mixture; and incubating the reaction mixture under conditions permitting extension of the one or more primers by the recombinant polymerase with the template for the incorporation of the extension nucleotides. The nucleic acid template can be selected from the group consisting of single-stranded DNA (ssDNA) templates, double-stranded DNA (dsDNA) templates, and RNA templates.

### 5. BRIEF DESCRIPTION OF DRAWINGS

The drawings are not necessarily to scale or exhaustive. Instead, emphasis is generally placed upon illustrating the principles of the inventions described herein. The accompanying drawings, which are incorporated in and constitute a part of this specification, illustrate several embodiments consistent with the disclosure and, together with the description, serve to explain the principles of the disclosure. In the drawings:

FIG. 1 depicts an SDS-PAGE analysis of the POL1 polymerase purification. Row 1 is supernatant. Row 2 is flow through. Rows 3 to 5 are wash with 10, 20 and 50 mM imidazole, respectively. Row 6 is a wash with 1 M NaCl. Rows 7 and 8 are elution steps with 125 mM imidazole. Rows 9 and 10 are elution with 250 mM imidazole. Row 11 is elution with 500 mM imidazole. Row 12 is POL1 comprising SEQ ID NO: 2 post-dialysis.

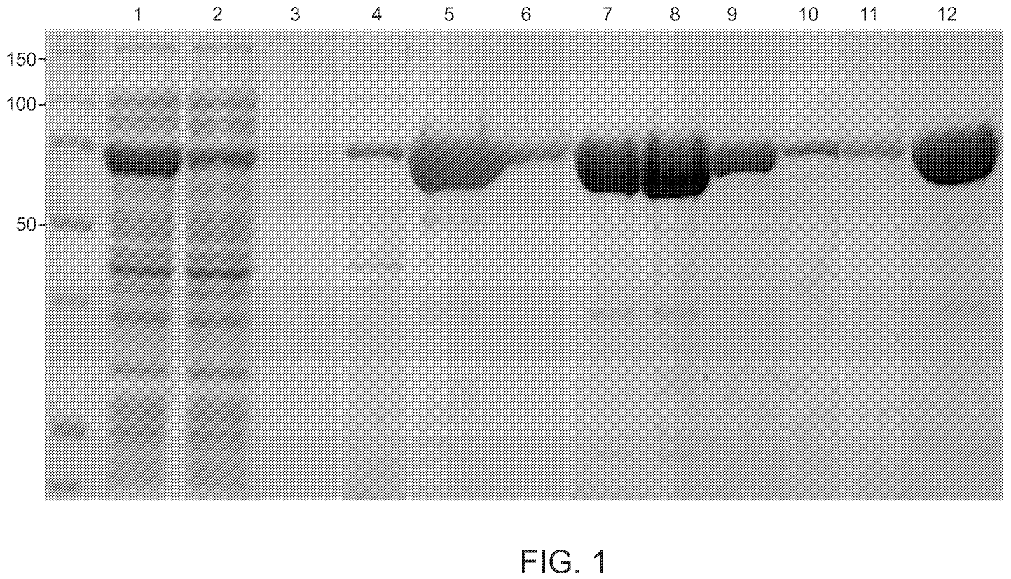

FIG. 2 depicts the results from an isothermal amplification experiment of POL1 polymerase at temperatures ranging from 42° C. to 65° C.

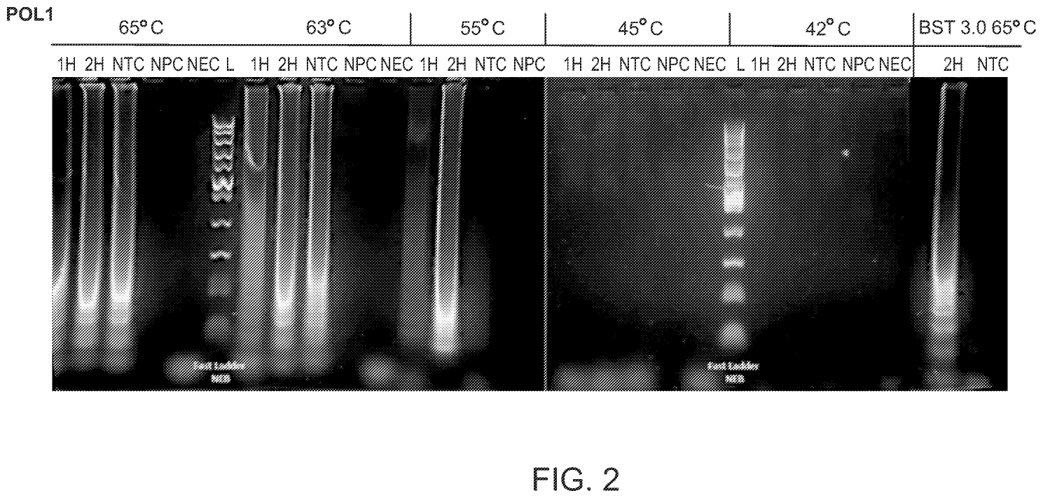

FIG. 3 depicts the results from a LAMP experiment using Buffer 4 and LAMP 6 primers.

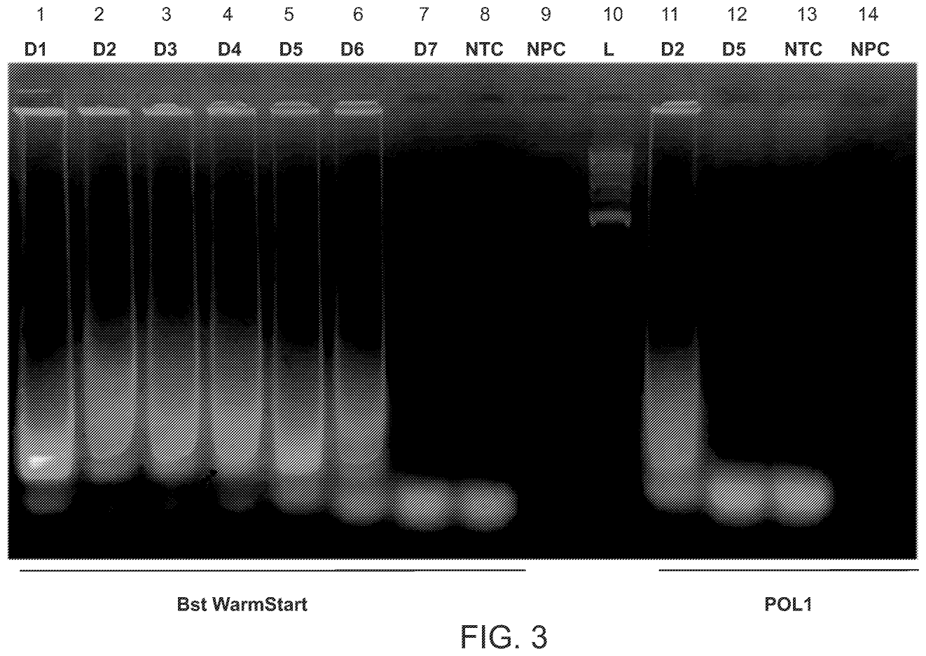

FIG. 4 depicts the thermal shift assay results for POL1 described in Example 4.

FIG. 5 depicts the results from LAMP reactions of POL1 polymerase at temperatures ranging from 51.6° C. to 56.0° C. described in Example 5.

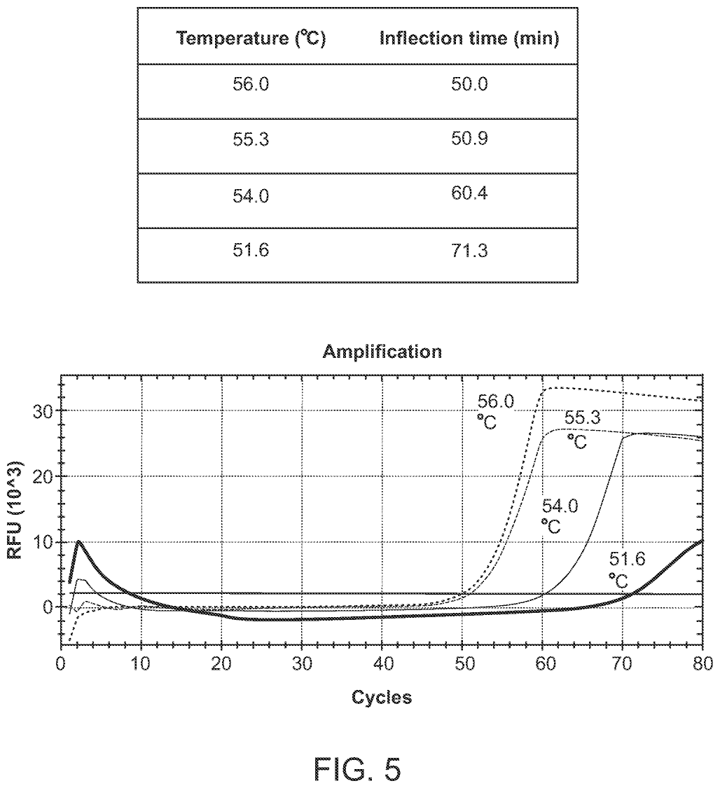

FIG. 6 depicts the results from LAMP reactions of POL1 polymerase at temperatures ranging from 56.9° C. to 70.0° C. described in Example 6.

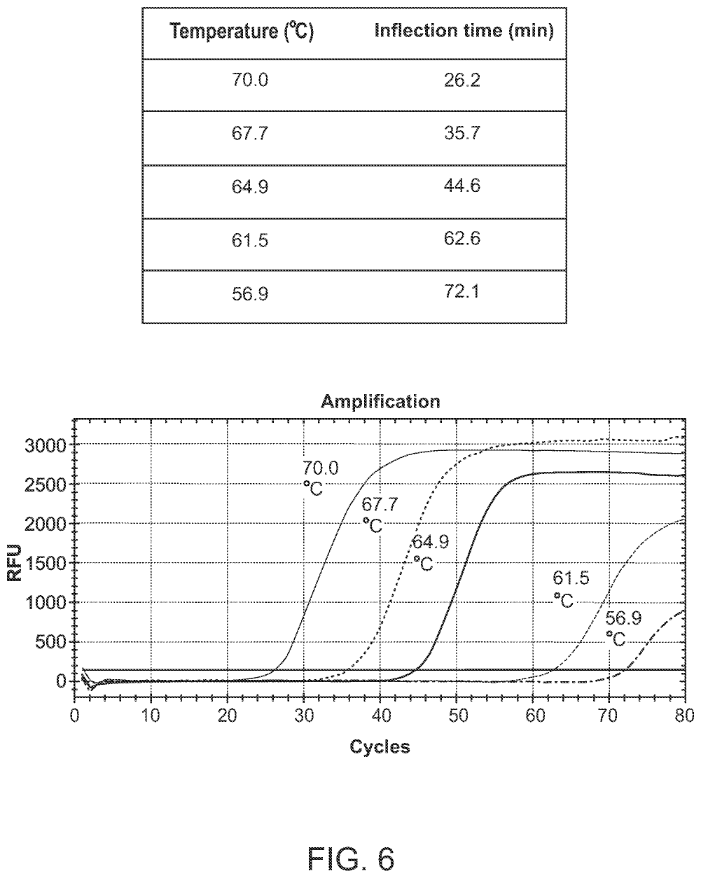

FIG. 7 depicts the results from LAMP reactions of POL1 polymerase at temperatures ranging from 70.0° C. to 85.0° C. described in Example 7.

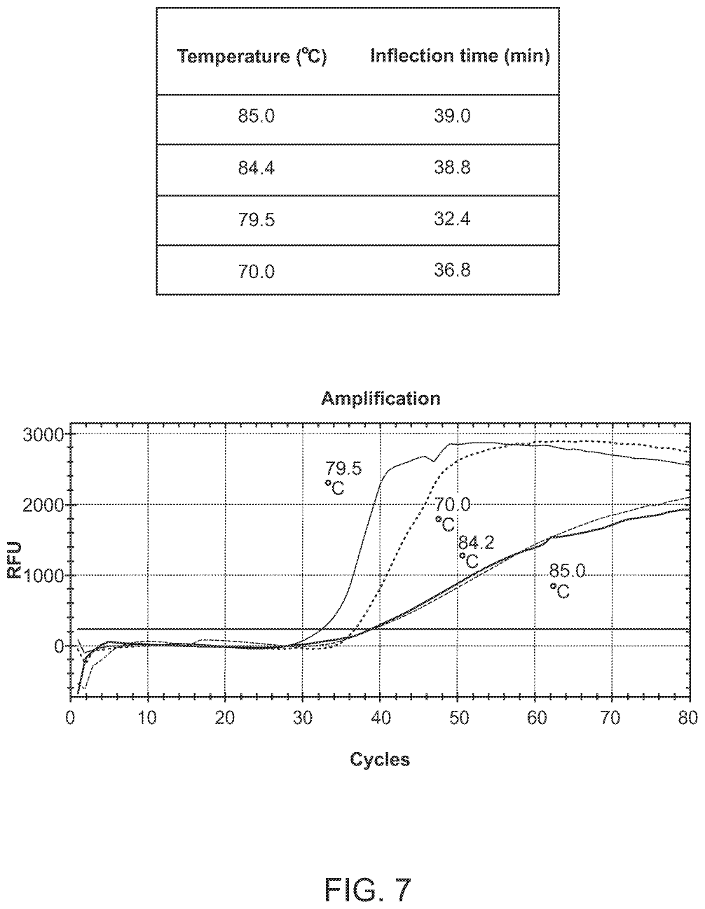

### 6. DETAILED DESCRIPTION

The disclosure relates to novel recombinant DNA polymerases and functional fragments thereof. The recombinant DNA polymerases disclosed herein possess properties that are particularly advantageous for nucleic acid amplification assays, such as isothermal amplification. For example, the DNA polymerase is active at a range of temperatures (i.e., 52° C. up to about 65° C.).

In some embodiments, the DNA polymerase can be active at a range of temperatures from about 51° C. to about 85° C.

Further disclosure relating to the recombinant DNA polymerase provided herein is provided below.

The specification is most thoroughly understood in light of the teachings of the references cited within the specification. Certain illustrative and preferred embodiments are described in detail herein. The embodiments within the specification should not be construed to limit the scope of the disclosure.

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “DNA” as used herein refers to deoxyribonucleic acid, a biopolymeric chain of predominantly deoxyribonucleotide residues linked generally by phosphodiester bonds.

As used herein, “dNTP” refers to deoxynucleotide triphosphate, e.g., dATP, dCTP, dGTP, dTTP, dUTP, and analogs thereof. As used herein, “nucleotide analogs” are molecules or ions comprising a base moiety other than the natural bases adenine (A), cytosine (C), guanine (G), thymine (T), or uracil (U), a sugar moiety identical or similar to deoxyribose, and at least one phosphate or multiple phosphate (e.g., diphosphate or triphosphate) moiety. The nucleotide analog is an analog of a specific nucleotide, in particular dATP, dCTP, dGTP, dTTP, or dUTP, when it comprises a triphosphate and a sugar moiety, the structure and configuration of both of which are suitable for incorporation into a nucleic acid double helix by a polymerase, and a base whose base pairing properties in a nucleic acid double helix and loci of incorporation by DNA polymerases in a nucleic acid double helix are most similar to one of the five previously listed nucleotides, with the exception that analogs of dTTP will generally also be analogs of dUTP and vice versa.

It is appreciated that certain features of the invention, which are, for clarity, described in the context of separate embodiments, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every sub-combination was individually and explicitly disclosed herein.

i. POL1 Polymerase

As described above, the inventors have developed novel recombinant polymerases that are suitable for nucleic acid amplification. The recombinant polymerases are DNA polymerases. The recombinant DNA polymerases may comprise an amino acid sequence having at least 70% sequence identity to SEQ ID NO: 1. The DNA polymerase may comprise an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater sequence identity to SEQ ID NO: 1.

The recombinant DNA polymerase may comprise an amino acid sequence having at least 70% sequence identity to SEQ ID NO: 2. SEQ ID NO: 2 is the C-terminus fragment of SEQ ID NO: 1. The recombinant DNA polymerase may comprise an amino acid sequence having at least 70% sequence identity to SEQ ID NO: 2. SEQ ID NO: 2 is the C-terminus fragment of SEQ ID NO: 1 with a mutation of Asp at position 365 of SEQ ID NO: 1 to Ala. Without wishing to be bound by theory or mechanism, it is believed that the C-terminus fragment contains the polymerase activity. The DNA polymerase may comprise an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater sequence identity to SEQ ID NO: 2.

The recombinant DNA polymerase may comprise about 1, about 2, about 3, about 4, about 5, about 6, about 7, about 8, about 9, about 10, about 11, about 12, about 13, about 14, about 15, about 16, about 17, about 18, about 19, about 20, or more amino acid alterations, e.g., amino acid substitutions or deletions, compared to SEQ ID NO: 1 or SEQ ID NO: 2. The amino acid substitution can be a conservative substitution or a non-conservative substitution, but preferably is a conservative substitution. A “conservative” amino acid substitution, as used herein, generally refers to substitution of one amino acid residue with another amino acid residue from within a recognized group, which can change the structure of the peptide yet biological activity of the peptide is substantially retained. Conservative substitutions of amino acids are known to those skilled in the art. Conservative substitutions of amino acids can include, but are not limited to, substitutions made amongst amino acids within the following groups: (a) M, I, L, V; (b) F, Y, W; (c) K, R, H; (d) A, G; (e) S, T; (f) Q, N; and (g) E, D. For example, a person of ordinary skill in the art would reasonably expect that an isolated replacement of a leucine with an isoleucine or valine, an aspartate with a glutamate, a threonine with a serine, or a similar replacement of an amino acid with a structurally related amino acid will not have a significant effect on the biological activity of the resulting molecule.

The recombinant DNA polymerases disclosed herein may be a variant or functional fragment of SEQ ID NO: 1 or SEQ ID NO: 2. Functional fragments are fragments of SEQ ID NO: 1 or SEQ ID NO: 2 that have DNA polymerase activity. Functional fragments of SEQ ID NO: 1 may be about 200 to about 943 amino acids in length. Functional fragments of SEQ ID NO: 1 may be at least about 200 amino acids in length, about 250 amino acids in length, about 300 amino acids in length, about 350 amino acids in length, about 400 amino acids in length, about 450 amino acids in length, about 500 amino acids in length, about 550 amino acids in length, about 600 amino acids in length, about 650 amino acids in length, about 700 amino acids in length, about 750 amino acids in length, about 800 amino acids in length, about 850 amino acids in length, about 900 amino acids in length, or up to about 943 amino acids in length.

Functional fragments of SEQ ID NO: 2 may be about 50 to about 654 amino acids in length. Functional fragments of SEQ ID NO: 2 may be at least about 50 amino acids in length, about 100 amino acids in length, about 150 amino acids in length, about 200 amino acids in length, about 250 amino acids in length, about 300 amino acids in length, about 350 amino acids in length, about 400 amino acids in length, about 450 amino acids in length, about 500 amino acids in length, about 550 amino acids in length, about 600 amino acids in length, or up to about 654 amino acids in length.

The functional fragments may be at least about 70%, about 75%, about 80%, about 85%, about 90%, about 95%, about 96%, about 97%, about 98%, about 99%, or about 100% identical to the amino acid sequence of SEQ ID NO: 1. The functional fragments may be at least about 70%, about 75%, about 80%, about 85%, about 90%, about 95%, about 96%, about 97%, about 98%, about 99%, or about 100% identical to the amino acid sequence of SEQ ID NO: 2.

The variant or functional fragment of SEQ ID NO: 1 or SEQ ID NO: 2 may retain at least about 65%, at least about 70%, at least about 75%, at least about 80%, at least about 90%, at least about 95%, at least about 100% of the activity.

Homology (e.g. sequence identity) may be assessed by any suitable method. However, for determining the degree of homology (e.g. identity) between sequences, computer programs that make multiple alignments of sequences are useful, for example Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) those described in Computational Molecular Biology, Lesk, Ed., Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:1 1-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose.

DNA polymerases generally have a temperature at which optimal polymerase activity is achieved. When the temperature deviates (e.g. decreases or increases) from the optimal temperature at which optimal activity is observed, there is generally a marked decrease in polymerase activity, even at only modest temperature deviations, meaning that many polymerases are only suitable for use in applications carried out in a fairly narrow temperature range.

The recombinant DNA polymerase described herein advantageously has activity over a range of temperatures. The recombinant DNA polymerase described herein can have activity at temperatures from about 52° C. to about 65° C. The recombinant DNA polymerase can have activity at about 52° C., about 53° C., about 54° C., about 55° C., about 56° C., about 57° C., about 58° C., about 59° C., about 60° C., about 61° C., about 62° C., about 63° C., about 64° C., about 65° C. At temperatures from about 52° C. to about 65° C., the recombinant DNA polymerase may have at least about 65%, at least about 70%, at least about 75%, at least about 80%, at least about 90%, at least about 95%, at least about 100% of its activity.

The recombinant DNA polymerase described herein can have activity at temperatures from about 51° C. to about 85° C., such as from about 52° C. to about 65° C. The recombinant DNA polymerase can have activity at about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., or about 85° C.

Suitable assays for analyzing DNA polymerase activity are known in the art. Such assays can be used to determine the recombinant DNA polymerase activity described herein at any given temperature. Exemplary assays for measuring DNA polymerase activity include, but are not limited to a single-nucleotide incorporation assay. In an exemplary single-nucleotide incorporation assay, a primer having about 19 nucleotides, which is labelled with a fluorophore at its 5′ end, is annealed to a template DNA strand consisting of 40 nucleotides; in the reaction set up the only dNTP present is dATP, thus the polymerase can extend the primer in the 5′-3′ direction only by one nucleotide at position 20 (as there is one T at the corresponding (complementary) position on the template strand); subsequent analysis on a denaturing polyacrylamide gel and scanning for the fluorophore labeled oligonucleotides shows the primer having 19 oligonucleotides and the primer extended by the nucleotide adenine thus consisting of 20 oligonucleotides; enzyme activity (i.e. polymerase activity) is determined by densitometric measurement of bands representing the extended primer (intensity 1) and the unextended primer (intensity 0); the relative incorporation rate is calculated as follows: incorporation [%]=intensity 1/(intensity 0+intensity 1)*100.

Other assays for measuring DNA polymerase activity include loop mediated isothermal amplification (LAMP), such as is described in Examples 5-7, and Crispr assays, such as is referred to in Example 8.

The recombinant DNA polymerase disclosed herein can be considered stable. In this context, a “stable” DNA polymerase means that the DNA polymerase retains substantial polymerase activity after exposure to a range of temperatures. For example, the recombinant DNA polymerase may retain at least about 65%, at least about 70%, at least about 75%, at least about 80%, at least about 90%, at least about 95%, at least about 100% of its activity.

The recombinant DNA polymerases disclosed herein may comprise high processivity. The term “processivity” as used herein refers to the ability of a DNA polymerase to carry out continuous DNA synthesis on a template DNA without frequent dissociation. It can be measured by the average number of nucleotides incorporated by a DNA polymerase on a single association/disassociation event. The recombinant DNA polymerases may have processivity comparable to commercially available DNA polymerases. The recombinant DNA polymerases may have processivity that is greater than about 5%, about 10%, about 15%, about 20%, about 25%, about 30%, about 40%, about 45%, about 50% or greater than commercially available DNA polymerases.

The recombinant DNA polymerases disclosed herein may function across a range of salt (e.g., NaCl) concentrations. The NaCl concentration at which the recombinant DNA polymerase exhibits its activity may be about 50 mM to 150 mM (e.g. 50, 55, 60, 65, 70, 75, 80, 85, 90, 95, 100, 105, 110, 115, 120, 130, 140, or 150 mM).

In some embodiments, the recombinant DNA polymerases disclosed herein may function in the presence of other salts (e.g., KCl, sodium acetate (NaAc), and KAc) across a range of concentrations. In some embodiments, the KAc concentration at which the recombinant DNA polymerase exhibits its activity can be about 25 mM to 150 mM (e.g. 25, 30, 35, 40, 45, 50, 55, 60, 65, 70, 75, 80, 85, 90, 95, 100, 105, 110, 115, 120, 130, 140, or 150 mM). In some embodiments, activity can be higher at salt concentrations from about 25 mM to about 100 mM.

The recombinant DNA polymerase may function across a range of MgSO4 concentrations, for example from about 6 mM to about 12 mM.

The recombinant DNA polymerase may function at pH values from about 6.8 to about 9.5.

In some embodiments, the recombinant DNA polymerase may function in the presence of dimethyl sulfoxide (DMSO), Triton X100, Tween 20, betaine, urea, bovine serum albumin (BSA), taurine, dithiothreitol (DTT), tris(2-carboxyethyl)phosphine (TCEP), threalose, NH₄SO₄, tetramethyl ammonium chloride (TMAC), tetra-n-propylammonium chloride (TPAC), polyethylene glycol (PEG), and/or 1,2-propanediol.

The recombinant DNA polymerases disclosed herein may have 3′ exonuclease activity. The recombinant DNA polymerase disclosed herein may lack 5′ exonuclease activity.

In embodiments wherein the recombinant DNA polymerase comprises SEQ ID NO: 1, the recombinant DNA polymerase can have both 3′-5′ exonuclease activity and 5′-3′ exonuclease activity.

In embodiments wherein the recombinant DNA polymerase comprises SEQ ID NO: 2, the recombinant DNA polymerase can lack both 3′-5′ exonuclease activity and 5′-3′ exonuclease activity.

ii. Nucleic Acids, Vectors, Cells

The recombinant DNA polymerases disclosed herein may be produced by recombinant techniques. Alternatively, the recombinant DNA polymerases can be chemically synthesized. Possible expression vectors include, but are not limited to, cosmids or plasmids, so long as the vector is compatible with the host cell used. The expression vectors can be “suitable for transformation of a host cell,” which means that the expression vectors contain a nucleic acid molecule encoding a recombinant DNA polymerase disclosed herein and regulatory sequences selected on the basis of the host cells to be used for expression, which are operatively linked to the nucleic acid molecule. The term “operatively linked” is intended to mean that the nucleic acid is linked to regulatory sequences in a way that allows for expression of the nucleic acid.

This disclosure further provides an expression vector containing a nucleic acid molecule encoding the recombinant DNA polymerase disclosed herein, a variant thereof, or a fragment thereof, and the necessary regulatory sequences for transcription and translation of the protein sequence encoded by the nucleic acid molecule.

Suitable regulatory sequences may be derived from a variety of sources, including bacterial, fungal, viral, mammalian, or insect genes and are well known in the art. Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription, may be incorporated into the expression vector.

The recombinant expression vectors disclosed herein may also contain a selectable marker gene that facilitates selection of host cells transformed or transfected with a recombinant DNA polymerase. Exemplary host cells include, but are not limited to, non-vertebrate cells, vertebrate cells, plant cells, yeast cells, or prokaryote cells. Examples of cells derived from vertebrate organisms that are useful as host cell lines include non-human embryonic stem cells or derivative thereof, for example avian EBX cells, monkey kidney CVI line transformed by SV40 sequences (COS-7, ATCC, CRL 1651), a human embryonic kidney line (293), baby hamster kidney cells (BHK, ATCC CCL 10), Chinese hamster ovary cells (CHO), mouse Sertoli cells (TM4), monkey kidney cells (CVI, ATCC CCL 70), African green monkey kidney cells (VERO-76, ATCC CRL-1 587), human cervical carcinoma cells (HeLa, ATCC CCL 2), canine kidney cells (MDCK, ATCC CCL 34), buffalo rat liver cells (BRL 3A, ATCC CRL 1442), human lung cells (W1 38, ATCC CCL 75), human liver cells (Hep G2, HB 8065), mouse mammary tumor cells (MMT 060562, ATCC CCL51), rat hepatoma cells (HTC, MI.5), YB2/0 (ATCC n° CRL1 662), NIH3T3, HEK and TRI cells.

Examples of prokaryotic cells that are useful as host cell lines include, but are not limited to, E. coli.

Recombinant expression vectors can be introduced into host cells to produce a transformed host cell. Transformation encompasses the introduction of nucleic acid (e.g., a vector) into a cell by one of many possible techniques known in the art. Suitable methods for transforming and transfecting host cells can be found in Sambrook et al., Molecular Cloning: A Laboratory Manual 4th ed. (2012) Cold Spring Harbor Laboratory Press, Cold Spring Harbor, NY and other laboratory textbooks. Suitable host cells include a wide variety of prokaryotic host cells and eukaryotic cells.

The recombinant DNA polymerases disclosed herein are preferably produced by recombinant techniques in a non-human host cell.

A method of producing the recombinant polymerases disclosed herein can comprise culturing a host cell comprising an expression vector that comprises an isolated nucleotide sequence encoding the recombinant DNA polymerase under conditions suitable for the expression of the nucleic acid encoding the recombinant DNA polymerase. The method may optionally comprise isolating or obtaining the recombinant DNA polymerase or protein from the host cell. The methods may further comprise a step of purification of the recombinant DNA polymerase. Any suitable purification technique can be used. Exemplary techniques include, but are not limited to, precipitation, ultrafiltration, dialysis, chromatography (e.g., size exclusion chromatography, ion-exchange chromatography, affinity chromatography), electrophoresis, and centrifugation.

In some embodiments, the recombinant DNA polymerase can contain an N-terminal or C-terminal polyhistidine (His tag), and the purification can include immobilized metal affinity chromatography. The His tag can optionally be subsequently removed from the recombinant DNA polymerase. A recombinant DNA polymerase comprising a His tag can retain essentially full activity.

The recombinant DNA polymerases can be formulated into a composition.

iii. Methods

Provided herein are methods of using a recombinant DNA polymerase disclosed herein. The recombinant DNA polymerases disclosed herein may be used to extend a nucleic acid (e.g., DNA) strand by one or more nucleotides. The recombinant DNA polymerases are particularly suitable for nucleic acid amplification or sequencing.

The recombinant DNA polymerases may be used in a molecular beacon assay, a strand displacement assay or in a single-nucleotide incorporation assay.

The recombinant DNA polymerases disclosed herein are particularly useful for assays that use a constant temperature (i.e., without thermocycling). Isothermal amplification reactions are particularly suitable for the recombinant DNA polymerases disclosed herein. Isothermal amplification reactions are typically performed at a constant temperature. Many isothermal amplification techniques are known in the art and include Loop mediated isothermal amplification (LAMP), rolling circle amplification (RCA), strand displacement amplification (SDA), multiple displacement amplification (MDA) and cross priming amplification (CPA).

Provided herein are methods of nucleotide polymerization using a recombinant DNA polymerase disclosed herein. A method may comprise providing a reaction mixture comprising a recombinant DNA polymerase disclosed herein, a template nucleic acid molecule, an oligonucleotide primer which is capable of annealing to a portion of the template nucleic acid molecule and one or more species of nucleotide (e.g. deoxynucleoside triphosphates, dNTPS) and incubating the reaction mixture under conditions whereby the oligonucleotide primer anneals to the template nucleic acid molecule and the DNA polymerase extends the oligonucleotide primer by polymerizing one or more nucleotides. Suitable conditions are known in the art. Preferably a constant temperature is used and preferred temperatures are set out elsewhere herein. Optionally, the generation of the polynucleotide product is detected (e.g. via gel electrophoresis).

Also provided herein are methods of amplifying a nucleic acid (e.g., DNA) using a recombinant DNA polymerase disclosed herein. The method may comprise providing to a reaction mixture comprising a recombinant DNA polymerase disclosed herein, a template nucleic acid molecule, an oligonucleotide primer(s) (e.g. 2 or more primers such as 2, 3, 4, 5 or 6 primers) which is capable of annealing to a portion of the template nucleic acid molecule acid molecule, and nucleotides (e.g. deoxynucleoside triphosphates, dNTPS) and incubating the reaction mixture under conditions whereby the oligonucleotide primer(s) anneals to the template nucleic acid molecule and the DNA polymerase extends the oligonucleotide primer(s) by polymerizing one or more nucleotides to generate a polynucleotide. Suitable conditions are well known in the art. Preferred methods of nucleic acid amplification are isothermal amplification methods.

The paragraph above identifies DNA as an exemplary nucleic acid that can be amplified by the method. In embodiments, the template nucleic acid can be selected from the group consisting of single-stranded DNA (ssDNA) templates, double-stranded DNA (dsDNA) templates, and RNA templates

Isothermal amplification is typically carried out at a constant temperature. The constant temperature used in the methods disclosed herein may be from about 52° C. to about 65° C. The isothermal amplification can be performed at about 52° C., about 53° C., about 54° C., about 55° C., about 56° C., about 57° C., about 58° C., about 59° C., about 60° C., about 61° C., about 62° C., about 63° C., about 64° C., or about 65° C.

In some embodiments, the constant temperature for isothermal amplification in the methods disclosed herein can be from about 51° C. to about 85° C., such as from at about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., or about 85° C.

Higher levels of amplification may be achieved using a divalent metal ion, e.g., Mg, Mn, Co, or Zn. The divalent metal ion may be provided as a salt that contains the metal ion and the conjugate base of an acid. Magnesium salts may comprise, e.g., magnesium chloride, magnesium acetate, magnesium sulfate, magnesium bromide, or magnesium iodide. Manganese salts may comprise, e.g., manganese chloride, manganese acetate, manganese sulfate, manganese bromide, or manganese iodide.

The recombinant DNA polymerases may be used in point-of-care diagnostic methods. The recombinant DNA polymerases disclosed herein may be used in whole genome amplification.

The recombinant DNA polymerases disclosed herein may be used in next-generation sequencing methods.

Further provided are compositions comprising a recombinant DNA polymerase described herein. The composition can further comprise a pharmaceutically acceptable excipient. The composition can further comprise a buffer. Optionally, compositions of the present invention further comprise one or more of the necessary reagents to carry out a nucleic acid amplification reaction (e.g. an isothermal amplification reaction), e.g. oligonucleotide primers capable of annealing to a region of the template DNA to be amplified and/or nucleotides (e.g. dNTPs). Typically compositions will be aqueous and buffered with a standard buffer such as Tris, HEPES, etc.

iv. Kits

Also disclosed herein are kits comprising a recombinant DNA polymerase disclosed herein, a buffer, and optionally a divalent metal, an extension nucleotide, a primer, a probe, a detergent, a detection agent, a dye, a fluorescent molecule, an anticoagulant, nucleoside triphosphates, or a cell lysis agent.

The kits can be used for a variety of applications. A preferred application is for nucleic acid amplification, such as isothermal amplification. One skilled in the art will recognize components of kits suitable for carrying out a method (or methods) of the present disclosure. For example, a kit may include one or more containers, each of which is suitable for containing one or more reagents or other means for nucleic acid amplification, and optionally instructions for carrying out one or more of the methods descried herein.

In some instances, the kit may also include one or more vials, tubes, bottles, dispensers, and the like, which are capable of holding one or more reagents needed to practice the present disclosure.

Instructions for the kits of the present disclosure may be affixed to packaging material, included as a package insert, and/or identified by a link to a website. While the instructions are typically written or printed materials, they are not limited to such. Any medium capable of storing such instructions and communicating them to an end user is contemplated by the present disclosure. Such media includes, but is not limited to, electronic storage media (e.g., magnetic discs, tapes, cartridges, chips), optical media (e.g., CD ROM), and the like. As used herein, the term “instructions” can include the address of an Internet site that provides the instructions. An example of this can include a kit that provides a web address where the instructions can be viewed and/or from which the instructions can be downloaded. In other instances, kits of the present disclosure may comprise one or more computer programs that may be used in practicing the methods of the present disclosure. For example, a computer program may be provided that takes the output from a microplate reader or a fluorescence spectrophotometer and prepares a calibration curve from the optical density observed in the wells and compares these densitometric or other quantitative readings to the optical density or other quantitative readings in wells.

In some embodiments, the kits may provide instructions, computer programs, or both relating to quantitation of amplified nucleic acid by a real-time PCR detection system.

### 7. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain methods in detail, the same will be more clearly understood by reference to the following examples, which are introduced for illustration only and not intended to be limiting.

### 8. EXAMPLES

### Example 1: Polymerase Purification

POL1 coding sequences were codon-optimized and synthesized by GeneScript. POL1 expression plasmids cloned into pET28a with N-terminal 6×His tagging (SEQ ID NO: 3) was transformed into E. coli NiCo21 (DE3) (New England Biolabs). For protein expression, a single clone was first cultured overnight in 10-mL liquid LB tubes and then inoculated into 1 L of fresh liquid LB (OD 600 0.1). Cells were grown with shaking at 200 rpm and 37° C. until the OD 600 reached 0.8, and IPTG was then added to a final concentration of 0.25 mM followed by further culture of the cells at 37° C. for about 3 h before the cell harvesting. Cells were resuspended in 20 mL of buffer A (50 mM Tris-HCl pH 8, 300 mM NaCl, 10% (v/v) glycerol, 10 mM imidazole, 1 mM DTT, 1 mM PMSF) and lysed by sonication after DNase I (100 ug/ml) and Lysozyme (100 μg/ml) treatment. Cell debris and insoluble particles were removed by centrifugation (15,000 rpm for 30 min). After centrifuging, the supernatant was loaded onto a batch affinity purification using a HisPur™ Ni-NTA resin (Thermo Fisher Scientific) equilibrated in buffer A with 10 mM imidazole. After three washing steps (buffer A plus 10-50 mM imidazole) an additional washing step with 1M NaCl was performed. Then, the elution by a gradient of buffer B (buffer A plus 125-500 mM imidazole) was carried out. The samples were then dialyzed against buffer containing 10 mM Tris HCl pH 8, 150 mM NaCl and 1 mM DTT.

### Example 2: POL1 Polymerase Isothermal Amplification

The isothermal amplification assay was performed using one primer pair designed specifically for each temperature ranges from 60-65° C. to 40° C. (Table 1).

TABLE 1
Isothermal amplification primers

| | |
|---|---|
| pET28a_700_Fw_60-65 | GAGCCATATTCAACGGG |
| | AAACGTCTTGCTC |
| | (SEQ ID NO: 4) |
| pET28a_700_Fw_50 | GAGCCATATTCAACGGG |
| | AAAC |
| | (SEQ ID NO: 5) |
| pET28a_700_Fw_40 | GAGCCATATTCAACG |
| | (SEQ ID NO: 6) |
| pET28a_700_Rv_60-65 | GATCCTGGTATCGGTCT |
| | GCGATTCCG |
| | (SEQ ID NO: 7) |
| pET28a_700_Rv_50 | GATCCTGGTATCGGTC |
| | TGC |
| | (SEQ ID NO: 8) |
| pET28a_700_Rv_40 | AGATCCTGGTATCGG |
| | (SEQ ID NO: 9) |

The reaction mixture consisted of 0.5 μM of each forward and reverse primers, 2.5 μl of 10×NEB4 buffer (New England Biolabs), 1.4 mM dNTPs (New England Biolabs), 0.56 ng/μl of POL1 or 0.32 U/μL of Bst 3.0 DNA polymerase (New England Biolabs) and 10 ng of pET28a dsDNA template (Novagen). The volume was adjusted to 25 μl with nuclease-free water. The reaction were subjected to an initial denaturation step at 95° C. for 3 minutes, after chilled at the amplification temperature POL1 or Bst 3.0 DNA polymerase (New England Biolabs) were added, followed by incubation at 65° C., 63° C., 55° C., 45° C. and 42° C. for 1 h, individually. Amplification products of 700 bp were analyzed using 2% agarose electrophoresis in Tris-acetic acid-EDTA (TAE) buffer (Bio-Rad). Gels were stained with SYBR gold (ThermoFisher) using a 1:10.000 dilution. Fast DNA ladder (New England Biolabs) was used as molecular weight marker. The gels were visualized by UV transillumination. Photo-documentation was done using a smartphone camera. The polymerase Bst 3.0 (New England Biolabs) was used as positive amplification reaction control. Non-template control (NTC), Non-Enzyme control (NEC) or Non-primers control (NPC) were used for each run to rule out the possibility of contamination of any of the reagents and specificity of polymerase activity.

### Example 3: POL1 LAMP Experiment

LAMP experiments were performed using primer sets specifically designed to target a highly conserved N gene of the SARS-CoV-2 viral genome. The following components comprised the LAMP assay: 1× final concentration of the NEB 4 buffer (New England Biolabs), 1.4 mM each of deoxyribonucleoside triphosphates (New England Biolabs). Along with the buffer components, a primer mix consisting of 0.2 μM F3 and B3, 1.6 μM forward Inner primer (FIP) and backward inner primer (BIP), and 0.8 μM of LoopF and LoopB were added to the reaction (Table 2). Finally, 0.56 ng/μl of POL1 or 0.32 U/μL of Bst DNA polymerase WarmStart (New England Biolabs) and 10⁸ to 100 copies/μl of 2019nCoV_N_Plasmid dsDNA template (IDT) were included to the final mix. The volume was adjusted to 25 μl with nuclease-free water. Negative controls (NTC and NPC) were included in all of the datasets. All of the LAMP assays were carried out in 0.2-mL PCR tubes in a Bio-Rad T100 thermal cycler PCR System at 65° C. for 60 min. Amplification products were analyzed using 2% agarose electrophoresis in Tris-acetic acid-EDTA (TAE) buffer (Bio-Rad). Gels were stained with SYBR gold (ThermoFisher) and Fast DNA ladder (New England Biolabs) was used as molecular weight marker. The gels were visualized by UV transillumination and photo-documentation was done using a smartphone camera.

TABLE 2
N gene LAMP primers

| | |
|---|---|
| N gene_F3 | TGGACCCCAAAATCAGCG |
| | (SEQ ID NO: 10) |
| N gene B3 | GGAACGCCTTGTCCTCGA |
| | (SEQ ID NO: 11) |
| N gene_FIP | CCACTGCGTTCTCCATTC |
| | TGGTAAATGCACCCCGCA |
| | TTACG |
| | (SEQ ID NO: 12) |
| N gene_BIP | CAACGTCGGCCCCAAGGT |
| | TTGGTCTTCCTTGCCATG |
| | TTGA |
| | (SEQ ID NO: 13) |
| N gene LF | TGAATCTGAGGGTCCAC |
| | CAAA |
| | (SEQ ID NO: 14) |
| N gene_LB | ACCCAATAATACTGCGT |
| | CTTGG |
| | (SEQ ID NO: 15) |

### Example 4: POL1 Thermal Shift

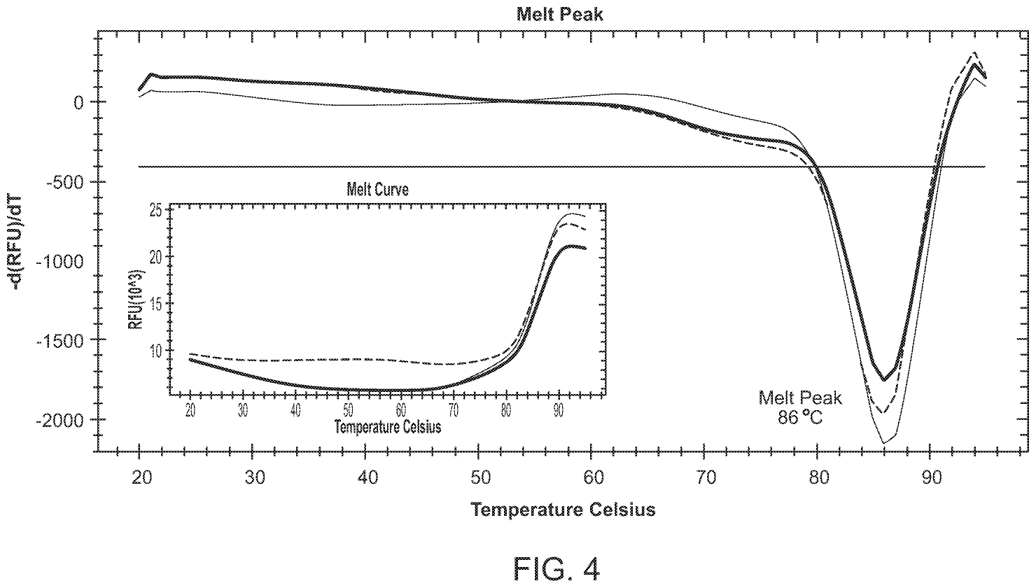

The thermal shift assay shows the thermal stability of an enzyme when it is exposed to an increasing temperature gradient. As the temperature rises, the enzyme denatures, exposing its hydrophobic residues. SYPRO™ reagent (Invitrogen #S6650) is able to bind to those residues and emit fluorescence. The inflection point of the curve is the thermal shift (TS) temperature of the enzyme. FIG. 4 shows the first derivative (main figure) of the fluorescence curve (inset). The reaction contained 10×SYPRO™ reagent and 13.5 ng POL1 in 20 μL final volume. The gradient range was 20-95° C. increasing 1° C./min. The experiment was run by triplicate in a CFX96 Touch Real-Time PCR Detection System (Bio-Rad). The data set was analyzed with CFX Maestro Software (Bio-Rad). The enzyme TS temperature was 86° C., demonstrating its thermostability.

### Example 5: LAMP Reactions of POL1 Polymerase at Temperatures Ranging from 51.6° C. to 56.0° C.

LAMP experiments were performed using primer sets specifically designed to target viral DNA from M13mp18 in the studied range, Table 3. The following components were used in the LAMP assay: 1× final concentration of the NEB 2 buffer (New England Biolabs), 1.4 mM each of deoxyribonucleoside triphosphates (New England Biolabs). In addition to the buffer components, a primer mix consisting of 0.2 μM F3 and B3, 1.6 μM forward Inner primer (FIP) and backward inner primer (BIP), and 0.8 μM of LoopF and LoopB were added to the reaction (Table 3). Finally, 56 ng/uL of POL1 and 10⁸ copies of M13mp18 single-stranded DNA template (New England Biolabs) were added to the reaction. The volume was adjusted to 25 μl with nuclease-free water. 800 nM SYTO™ 9 Green Fluorescent Nucleic Acid Stain (Invitrogen) were used as a reporter. The reaction was carried out in a CFX96 Touch Real-Time PCR Detection System (Bio-Rad) at temperatures ranging from 51.6° C. to 56.0° C. Fluorescent signal was measured in FAM channel every minute for 80 minutes. The amplification data was analyzed with CFX Maestro Software (Bio-Rad).

TABLE 3
M13_50 LAMP primers

| | |
|---|---|
| M13_50_F3 | TATTGTCTGTGCCAC |
| | (SEQ ID NO: 16) |
| M13_50_B3 | AAACTATCGGCCTTG |
| | (SEQ ID NO: 17) |
| M13_50_FIP | CACACGACCAGTAAT |
| | AATTTCAGGTCAGAA |
| | GG |
| | (SEQ ID NO: 18) |
| M13_50_BIP | GACGATTGAGCGTCA |
| | ACAATATTACCGCCA |
| | (SEQ ID NO: 19) |
| M13_50_LF | GCCAACAGAGATAGAAC |
| | (SEQ ID NO: 20) |
| M13_50_LB | GTAGGTATTTCCATGAGC |
| | (SEQ ID NO: 21) |

### Example 6: LAMP Reactions of POL1 Polymerase at Temperatures Ranging from 56.9° C. to 70.0° C.

LAMP experiments were performed using primer sets specifically designed to target viral DNA from M13mp18 in the studied range, Table 4. The following components were used in the LAMP assay: 1× final concentration of the NEB 4 buffer (New England Biolabs), 1.4 mM each of deoxyribonucleoside triphosphates. Along with the buffer components, a primer mix consisting of 0.2 μM F3 and B3, 1.6 μM forward Inner primer (FIP) and backward inner primer (BIP), and 0.8 μM of LoopF and LoopB were added to the reaction (Table 4). Finally, 45 ng/uL POL1 and 10⁸ copies of M13mp18 RF I double-stranded DNA template (NEB) were added to the reaction. The volume was adjusted to 25 μl with nuclease-free water. 500 nM SYTO™ 9 Green Fluorescent Nucleic Acid Stain (Invitrogen) was used as a reporter. The reaction was carried out in a CFX96 Touch Real-Time PCR Detection System (Bio-Rad) at temperatures ranging from 56.9° C. to 70.0° C. and fluorescent signal was measured in FAM channel every minute for 80 minutes. The amplification data was analyzed with CFX Maestro Software (Bio-Rad).

TABLE 4
M13_60 LAMP primers

| | |
|---|---|
| M13_60_F3 | GCAGCACATCCCCCTTTC |
| | (SEQ ID NO: 22) |
| M13_60_B3 | CGTGCATCTGCCAGTTTGA |
| | (SEQ ID NO: 23) |
| M13_60_FIP | GGCTGCGCAACTGTTGGGA |
| | ACCAGCTGGCGTAATA |
| | GCG |
| | (SEQ ID NO: 24) |
| M13_60_BIP | CTTTGCCTGGTTTCCGGCA |
| | CACGACAGTATCGGCCT |
| | CAG |
| | (SEQ ID NO: 25) |
| M13_60_LF | ATCGGTGCGGGCCTCTT |
| | (SEQ ID NO: 26) |
| M13_60_LB | CAGAAGCGGTGCCGGAAA |
| | G |
| | (SEQ ID NO: 27) |

### Example 7: LAMP Reactions of POL1 Polymerase at Temperatures Ranging from 70.0° C. to 85.0° C.

LAMP experiments were performed using primer sets specifically designed to target human Beta-actin, Table 5. The following components were used in the LAMP assay: 1× final concentration of 10 mM Tris pH 8.8, 25 mM KAc, 8 mM MgSO4 and 1 mM DTT buffer and 1.4 mM each of deoxyribonucleoside triphosphates (New England Biolabs). In addition to the buffer components, a primer mix consisting of 0.2 μM F3 and B3, 1.6 μM forward Inner primer (FIP) and backward inner primer (BIP), and 0.8 μM of LoopF and LoopB were added to the reaction (Table 5). Finally, 45 ng/uL of POL1 and 36 ng Human Genomic DNA template (Promega) were added to the reaction. The volume was adjusted to 25 μl with nuclease-free water. 500 nM SYTO™ 9 Green Fluorescent Nucleic Acid Stain (Invitrogen) was used as a reporter. The reaction was carried out in a CFX96 Touch Real-Time PCR Detection System (Bio-Rad) at temperatures ranging from 70.0° C. to 85.0° C. and fluorescent signal was measured in FAM channel every minute for 80 minutes. The amplification data was analyzed with CFX Maestro Software (Bio-Rad).

TABLE 5
Beta-actin LAMP primers

| | |
|---|---|
| Bact_F3 | GCGTGATGGTGGGCATG |
| | (SEQ ID NO: 28) |
| Bact_B3 | TCTGGGTCATCTTCTCGCG |
| | (SEQ ID NO: 29) |
| Bact_FIP | TGCCGTGCTCGATGGGGT |
| | AGAAGGATTCCTATGTGG |
| | GCG |
| | (SEQ ID NO: 30) |
| Bact_BIP | CTCCCGAGGAGCACCCCG |
| | GTTGGCCTTGGGGTTCAG |
| | (SEQ ID NO: 31) |
| Bact_LF | GCCTCTCTTGCTCTGGGC |
| | (SEQ ID NO: 32) |
| Bact_LB | CTGCTGACCGAGGCCCC |
| | (SEQ ID NO: 33) |

### Example 8: Retrotranscriptase Activity Test of POLL

Retro-transcriptase activity of POL1 was tested by performing RT-LAMP with or without the addition of WarmStart® RTx Reverse Transcriptase (New England Biolabs) and decreasing amounts of RNA target from 10³ to 10⁶ copies per reaction (Quantitative genomic RNA from Severe acute respiratory syndrome-related coronavirus 2 strain 2019-nCoV/USA-WA1/2020, ATCC). The following components were used in the LAMP assay: 1× final concentration of 10 mM Tris pH 8.8, 25 mM KAc, 8 mM MgSO4 and 1 mM DTT buffer and 1.4 mM each of deoxyribonucleoside triphosphates (New England Biolabs). In addition to the buffer components, a primer mix consisting of 0.2 μM F3 and B3, 1.6 μM forward Inner primer (FIP) and backward inner primer (BIP), and 0.8 μM of LoopF and LoopB were added to the reaction (Table 2). Finally, 56 ng/uL POL1 and the RNA template were added to the reaction. 10⁶ copies per reaction of DNA SARS-CoV-2 (New England Biolabs) was added as a positive control of the reaction. Non-template control (NTC) was used as a negative control. The volume was adjusted to 25 μl with nuclease-free water. The reaction was carried out in a CFX96 Touch Real-Time PCR Detection System (Bio-Rad) at 70.0° C. for 120 minutes. The formation of specific product was confirmed by a standard Crispr assay. Results are summarized in Table 6. POL1 was active even with 10⁴ RNA copies without the addition of extra retrotranscriptase.

TABLE 6
Crispr results for Retrotranscriptase activity test of POL1

| Retrotran- | | | | | | |
|---|---|---|---|---|---|---|
| scriptase | | RNA | | | | DNA |
| addition | NTC | 10³ | 10⁴ | 10⁵ | 10⁶ | 10⁶ |
| No | Negative | Negative | Positive | Positive | Positive | Positive |
| Yes | Negative | Positive | Positive | Positive | Positive | Positive |

| SEQ ID | | |
|---|---|---|
| NO: | Description | Sequence |
| SEQ ID | Amino acid | MASLVVLIDGHSLAYRAYYALLRQG |
| NO: 1 | sequence | LQTRKGEPTHAVYGETSMLLRVWRE |
| | of full- | HQPTYMAVAFDVGRTFRHEAFAEYK |
| | length | ATRAERPSDEDAQLARIRQIIAAFA |
| | POL1 | IPALTAEGFEADDVIGTAATQALAR |
| | polymerase | GMEVLIVTGDTDTFQLIRPGLRVLT |
| | | SRRHEDDVVIYDVEAIRERYGLEPS |
| | | QLVDLKALTGDPSDNIPGVRGIGER |
| | | TATELLKRYGSLENLYAHLDEIQPE |
| | | RVRRLLEAGREQAFLSKRLAQIRTD |
| | | VPLTIDWEACRVGRYDRAAVLALFQ |
| | | ELEFRSLIPRLPDGAAPEEAPAAPE |
| | | AASGPTGQPMLFPEPLGPAVTRPIA |
| | | MPPLRAEGLPRPTEATVVTDEAGLA |
| | | ALQEALARAPRFAFDVETDTLAPMR |
| | | ANPVGLAFAVEDGRGYYVPVGHRDG |
| | | SPQLPLPRVLDALRPFLADPGRPKV |
| | | AHHAKYDMLVLARHGVMVQGLAFDT |
| | | MVAEWLLNPTAHGLSLKHLAWLRLG |
| | | VEMTTIEQLIGKGKGQRSFADVPTS |
| | | AAAPYAAADADLTLRIARVQEEELR |
| | | RQGLWDLFAQVEIPLIPVLVRMELA |
| | | GVLIDVALLQQMSKEFERRIQALAQ |
| | | EIYRWVGYAFNLDSPRQLSDALEGK |
| | | LQLPRANVPRTGTGMYSTSSEVLEG |
| | | LRGAHPVIDLILEYRQLAKLKSTYI |
| | | DALPRMIHPATGRIHPSYHQTGTVT |
| | | GRISASDPNIQNIPIRTELGKQIRR |
| | | AFIAPPGSVLLSADYSQIELRILAH |
| | | ITRDPGLIAAFQAGEDIHRATAARA |
| | | FGIPPEQVTEEQRNFAKRINYGLIY |
| | | GMSAHGLAQQLGISRREAERFIEQY |
| | | FAAFPRVRAYIEAIKARAARQGYVE |
| | | TLLGRRRYFPELKPAGGEGLPRRVP |
| | | EAVRRAAEREAINAPIQGSAADILK |
| | | LAMIRIDRAMQEQGLRTRMIMQVHD |
| | | ELVFEVPEDEVEAVQGLVAEAMRTA |
| | | YPLAVPLEVELHMGPCWS |
| SEQ ID | Amino acid | MAPEEAPAAPEAASGPTGQPMLFPE |
| NO: 2 | sequence | PLGPAVTRPIAMPPLRAEGLPRPTE |
| | of | ATVVTDEAGLAALQEALARAPREAF |
| | truncated | AVETDTLAPMRANPVGLAFAVEDGR |
| | (C- | GYYVPVGHRDGSPQLPLPRVLDALR |
| | terminus) | PFLADPGRPKVAHHAKYDMLVLARH |
| | POL1 | GVMVQGLAFDTMVAEWLLNPTAHGL |
| | polymerase | SLKHLAWLRLGVEMTTIEQLIGKGK |
| | | GQRSFADVPTSAAAPYAAADADLTL |
| | | RIARVQEEELRRQGLWDLFAQVEIP |
| | | LIPVLVRMELAGVLIDVALLQQMSK |
| | | EFERRIQALAQEIYRWVGYAFNLDS |
| | | PRQLSDALFGKLQLPRANVPRTGTG |
| | | MYSTSSEVLEGLRGAHPVIDLILEY |
| | | RQLAKLKSTYIDALPRMIHPATGRI |
| | | HPSYHQTGTVTGRISASDPNIQNIP |
| | | IRTELGKQIRRAFIAPPGSVLLSAD |
| | | YSQIELRILAHITRDPGLIAAFQAG |
| | | EDIHRATAARAFGIPPEQVTEEQRN |
| | | FAKRINYGLIYGMSAHGLAQQLGIS |
| | | RREAERFIEQYFAAFPRVRAYIEAI |
| | | KARAARQGYVETLLGRRRYFPELKP |
| | | AGGEGLPRRVPEAVRRAAEREAINA |
| | | PIQGSAADILKLAMIRIDRAMQEQG |
| | | LRTRMIMQVHDELVFEVPEDEVEAV |
| | | QGLVAEAMRTAYPLAVPLEVELHMG |
| | | PCWS |

### 9. SEQUENCE LISTING

1. A recombinant polymerase comprising an amino acid sequence having at least about 70% sequence identity to SEQ ID NO: 1 or SEQ ID NO: 2.

2. The recombinant polymerase of claim 1, wherein the recombinant polymerase comprises an amino acid sequence having at least about 90% sequence identity to SEQ ID NO: 1 or SEQ ID NO: 2.

3. The recombinant polymerase of claim 1, wherein the recombinant polymerase comprises an amino acid sequence having at least 99% sequence identity to SEQ ID NO: 2.

4. The recombinant polymerase of claim 1, wherein the recombinant polymerase is a DNA polymerase.

5. The recombinant polymerase of claim 1, wherein the recombinant polymerase is capable of 3′-5′ exonuclease activity and 5′-3′ exonuclease activity.

6. The recombinant polymerase of claim 1, wherein the recombinant polymerase does not have 5′-3′ exonuclease activity and 3′-5′ exonuclease activity.

7. The recombinant polymerase of claim 1, wherein the recombinant polymerase has activity at a temperature of about 51° C. to about 85° C.

8. The recombinant polymerase of claim 1, wherein the recombinant polymerase is suitable for use in isothermal amplification.

9. A composition comprising the recombinant polymerase of claim 1.

10. An isolated nucleic acid comprising a nucleotide sequence that encodes the recombinant polymerase according to claim 1.

11. An expression vector comprising the isolated nucleic acid of claim 10.

12. A host cell comprising the expression vector of claim 11.

13. A method of producing a recombinant polymerase, the method comprising: culturing the host cell of claim 12 under conditions suitable for expression of the isolated nucleic acid encoding the recombinant polymerase.

14. A kit comprising:
(a) the recombinant polymerase according to claim 1; and
(b) a buffer.

15. A method of amplifying a nucleic acid template, the method comprising:
(a) contacting the nucleic acid template with the recombinant polymerase according to claim 1, one or more primers that specifically bind to the template, and extension nucleotides to form a reaction mixture; and
(b) incubating the reaction mixture under conditions permitting extension of the one or more primers by the recombinant polymerase with the template for the incorporation of the extension nucleotides.

16. The method of claim 15, wherein the nucleic acid template is selected from the group consisting of single-stranded DNA (ssDNA) templates, double-stranded DNA (dsDNA) templates, and RNA templates.

17. The kit of claim 14, further comprising:
(c) a divalent metal, an extension nucleotide, a primer, a probe, a detergent, a detection agent, a dye, a fluorescent molecule, an anticoagulant, nucleoside triphosphates, a cell lysis agent, a salt, Triton X100, Tween 20, betaine, urea, bovine serum albumin (BSA), taurine, dithiothreitol (DTT), tris(2-carboxyethyl)phosphine (TCEP), trehalose, NH₄SO₄, tetramethyl ammonium chloride (TMAC), tetra-n-propylammonium chloride (TPAC), polyethylene glycol (PEG), 1,2-propanediol, or two or more thereof.

18. The kit of claim 17, wherein the salt is NaCl, KCl, sodium acetate (NaAc), KAc, MgSO₄, dimethyl sulfoxide (DMSO), or two or more thereof.
